(19) United States
(12) Patent Application Publication
(10) Pub. No.: US 2016/0159710 A1
(43) Pub. Date: Jun. 9, 2016

(54) DISPROPORTIONATION OF HYDROCARBONS USING SOLID ACID CATALYSTS

(71) Applicant: UOP LLC, Des Plaines, IL (US)

(72) Inventors: Stuart Smith, Lake Zurich, IL (US); Alakananda Bhattacharyya, Glen Ellyn, IL (US); Tom N. Kalnes, LaGrange, IL (US); Mary Wier, Schaumburg, IL (US); Paul T. Barger, Arlington Heights, IL (US)

(21) Appl. No.: 14/562,390

(22) Filed: Dec. 5, 2014

(51) Int. Cl. C07C 6/10

(52) U.S. Cl. CPC C07C 6/10; C07C 2527/125; C07C 2523/42

(57) ABSTRACT

A hydrocarbon disproportionation process is described. The process includes contacting a hydrocarbon feed in a disproportionation reaction zone with a disproportionation catalyst in the presence of hydrogen and an added chloride promoter under disproportionation conditions including to obtain disproportionation products, wherein the disproportionation catalyst comprises a solid catalyst comprising a refractory inorganic oxide having a metal halide dispersed thereon.

### BACKGROUND OF THE INVENTION

The isomerization of light naphtha has become an important process for the upgrading of petroleum refiners' gasoline pool. The removal of lead antiknock additive from gasoline and the rising demands of high-performance internal-combustion engines increased the need for “octane,” or knock resistance, in the gasoline pool. Isomerization processes have been used to improve the low octane numbers (RON) of light straight run naphtha. Isomerization processes involve reacting one mole of a hydrocarbon (e.g., normal pentane) to form one mole of an isomer of that specific hydrocarbon (e.g., isopentane), as shown in FIG. 1. The total number of moles remains the same throughout this process, and the product has the same number of carbons as the reactant.

The Reid vapor pressure (RVP) of gasoline has been utilized by the Environmental Protection Agency as a means of regulating volatile organic compounds emissions by transportation fuels and for controlling the formation of ground level ozone. As these regulations become more stringent and as more ethanol (which has a high vapor pressure) is blended into gasoline, C₅ paraffins need to be removed from the gasoline pool. Moreover, the need to remove components may also extend to some C₆ paraffins. This may result in refiners being oversupplied with C₅ paraffins and possibly C₆ paraffins.

There is a need for processes which can turn lower value hydrocarbons into higher value hydrocarbons.

### SUMMARY OF THE INVENTION

One aspect of the invention is a paraffin disproportionation process. In one embodiment, the process includes contacting a hydrocarbon feed in a disproportionation reaction zone with a disproportionation catalyst in the presence of hydrogen and an added chloride promoter under disproportionation conditions to obtain disproportionation products, wherein the disproportionation catalyst comprises a solid catalyst comprising a refractory inorganic oxide having a metal halide dispersed thereon.

### BRIEF DESCRIPTION OF THE DRAWINGS

FIG. 1 illustrates the isomerization reaction of n-pentane.

FIG. 2 illustrates the disproportionation reaction of iso-pentane.

FIG. 3 is a schematic of one embodiment of the process of present invention.

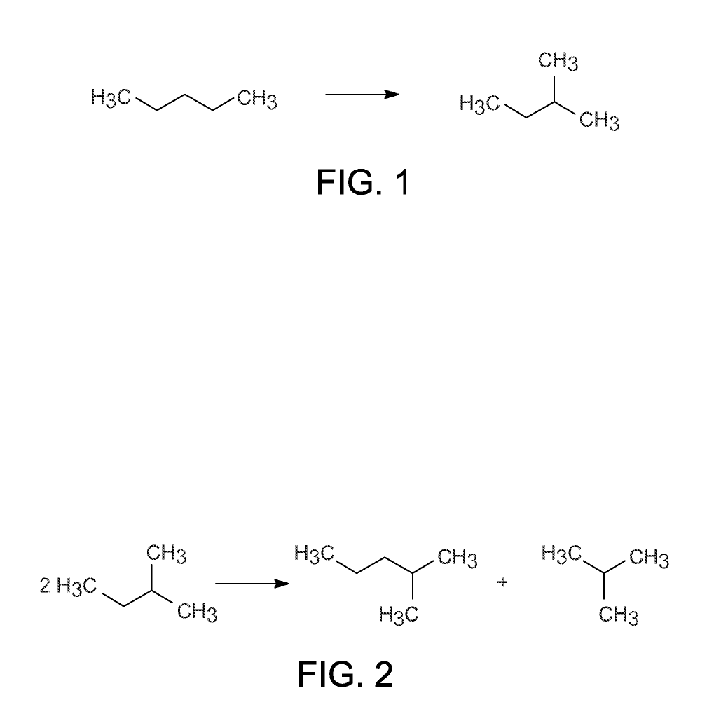

### DETAILED DESCRIPTION OF THE INVENTION

Disproportionation reactions offer a possible solution to the problem of excess C₅ and C₆ paraffins. The disproportionation of paraffins (e.g., isopentane (iC₅)) involves reacting two moles of hydrocarbon to form one mole each of two different products, one having a carbon count greater than the starting material and the other having a carbon count less than the starting material, as shown in FIG. 2. The total number of moles in the system remains the same throughout the process, but the products have different carbon counts from the reactants. For a feed of Cₓ, the disproportionation products include Cₓ⁺ hydrocarbons and Cₓ⁻ hydrocarbons.

Disproportionation reactions differ from cracking reactions in which one mole of a hydrocarbon forms two moles of product, each with a lower carbon number than the starting material.

As used herein, Cₓ means hydrocarbon molecules that have “X” number of carbon atoms, Cₓ⁺ means hydrocarbon molecules that have “X” and/or more than “X” number of carbon atoms, and Cₓ⁻ means hydrocarbon molecules that have “X” and/or less than “X” number of carbon atoms.

As used herein, the term “stream” can include various hydrocarbon molecules and other substances. Moreover, the term “stream comprising Cₓ hydrocarbons” can include a stream comprising hydrocarbon with “x” number of carbon atoms, suitably a stream with a majority of hydrocarbons with “x” number of carbon atoms and preferably a stream with at least 75 wt % hydrocarbons with “x” number of carbon atoms. Where a stream is identified as comprising Cₓ and C_{x′} hydrocarbons, the stream preferably has at least 75 wt % hydrocarbons with “x” and “x′” number of carbon atoms. Moreover, the term “stream comprising Cₓ₊ hydrocarbons” can include a stream comprising a majority of hydrocarbon with more than or equal to “x” carbon atoms and suitably less than 10 wt % and preferably less than 1 wt % hydrocarbon with x−1 carbon atoms. Lastly, the term “Cₓ₋stream” can include a stream comprising a majority of hydrocarbon with less than or equal to “x” carbon atoms and suitably less than 10 wt % and preferably less than 1 wt % hydrocarbon with x+1 carbon atoms.

As used herein, the term “zone” can refer to an area including one or more equipment items and/or one or more sub-zones. Equipment items can include one or more reactors or reactor vessels, heaters, exchangers, pipes, pumps, compressors, controllers and columns. Additionally, an equipment item, such as a reactor, dryer, or vessel, can further include one or more zones or sub-zones.

As used herein, the term “about” means within 10% of the value, or within 5%, or within 1%.

It has been found that solid acid catalysts can be used to catalyze hydrocarbon disproportionation reactions. The catalyst comprises a refractory inorganic oxide having a metal halide dispersed thereon. There can optionally be a Group VIII metal component dispersed thereon. The reaction takes place in the presence of hydrogen and a chloride promoter.

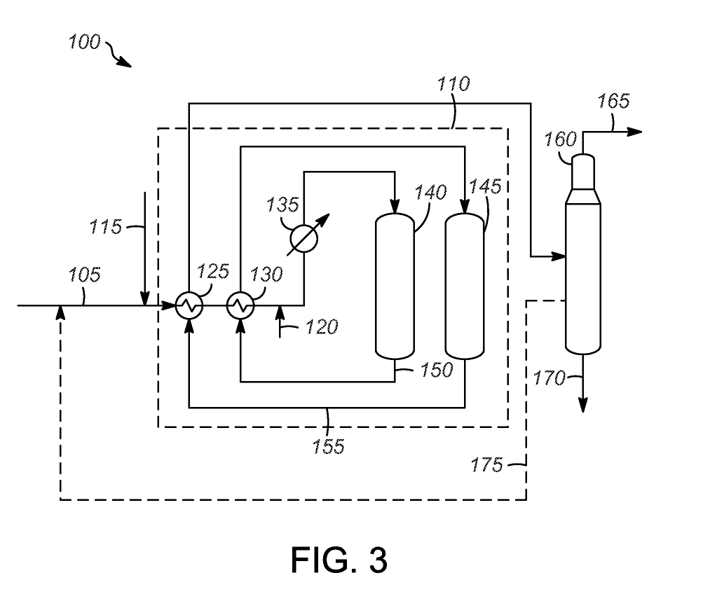

FIG. 3 illustrates one embodiment of the process 100. The hydrocarbon feed 105 is introduced into the disproportionation reaction zone 110.

The hydrocarbon feed 105 comprises hydrocarbons capable of disproportionation. One example of a suitable hydrocarbon feed 105 comprises alkanes having 4 to 7 carbon atoms. These may be contained in streams from petroleum refining, synthetic-fuel production, and biomass conversion, for example. Suitable streams from petroleum refining include, but are not limited to, natural gas liquids (NGLs), liquefied petroleum gas (LPGs), light straight-run naphtha, light naphtha, light natural gasoline, light reformate, light raffinate from aromatics extraction, light cracked naphtha, butanes, normal-butane concentrate, field butanes and the like. An especially preferred feedstock is light straight-run naphtha, containing more than 50% of C₅ and C₆ paraffins with a high concentration of low-octane normal paraffins. The light straight-run naphtha and other feedstocks also may contain naphthenes, aromatics, olefins, and hydrocarbons heavier than C₆. The olefin content should be limited to a maximum of 10% and the content of hydrocarbons heavier than C₆ to 20% for effective control of hydrogen consumption, cracking reactions, heat of reaction and catalyst activity.

The hydrocarbon feed 105 may need to be treated to remove sulfur-, nitrogen- and oxygen-containing compounds to prevent them from poisoning the disproportionation catalyst. The feedstock may be treated by any method that will remove water, sulfur-, nitrogen-, and oxygen-containing compounds. Sulfur may be removed from the feed stream by hydrotreating. Adsorption systems for the removal of sulfur-, nitrogen- and oxygen-containing compounds and water from hydrocarbon streams are well known to those skilled in the art.

The disproportionation reaction takes place in the presence of hydrogen 115 which has been shown to increase the catalyst stability significantly. The hydrogen 115 can be introduced into the system dissolved in the hydrocarbon feed 105 or directly into the disproportionation reaction zone 110. The hydrogen 115 may be supplied totally from outside the process or supplemented by hydrogen recycled to the feed after separation from reactor effluent. Light hydrocarbons and small amounts of inerts such as nitrogen and argon may be present in the hydrogen. Water should be removed from hydrogen supplied from outside the process, preferably by an adsorption system as is known in the art.

The mole ratio of hydrogen 115 to hydrocarbon feed 105 is in the range of about greater than 0:1 to about 2:1, or 0:1 to about 1.5:1, or 0:1 to about 0.75:1, or 0:1 to about 0.5:1, or 0:1 to about 0.3:1, or 0:1 to about 0.1:1, or 0:1 to about 0.05:1, or about 0:1 to about 0.02:1, or 0:1 to about 0.01:1, or 0.01:1 to about 0.05:1.

The disproportionation reaction takes place in the presence of an added chloride promoter 120. The chloride promoter typically comprises carbon tetrachloride, tetrachloroethylene, propyldichloride, butylchloride, chloroform, 2-chloro-2-methylpropane, 2-chloropropane, 2-chloro-2-methylbutane, 2-chloropentane, 1-chlorohexane, 3-chloro-3-methylpentane, 2-chlorobutane, or combinations thereof.

The chloride concentration of the added chloride promoter is typically in the range of greater than 0 to about 5000 ppm, and it typically ranges from about 100 ppm to about 5000 ppm, or about 200 ppm to about 5000 ppm, or about 400 ppm to about 5000 ppm, or about 600 ppm to about 5000 ppm, or about 800 ppm to about 5000 ppm, or about 1000 ppm to about 5000 ppm, or about 1200 ppm to about 5000 ppm, or about 1400 ppm to about 5000 ppm, or about 1600 ppm to about 5000 ppm. The chloride promoter 120 can be added to the hydrocarbon feed 105, for example being dissolved in the hydrocarbon feed, or directly to the disproportionation reaction zone 110.

The mole ratio of hydrogen to chloride from the added chloride promoter is in the range of greater than 0:1 to about 5000:1, or 0:1 to about 2500:1, or 0:1 to about 1000:1, or 0:1 to about 750:1, or 0:1 to about 500:1, or 0:1 to about 250:1, or 0:1 to about 225:1, or 0:1 to about 200:1, or 0:1 to about 175:1, or 0:1 to about 150:1, or 0:1 to about 125:1, or 0:1 to about 100:1, or 0:1 to about 75:1, or 0:1 to about 50:1, or 0:1 to about 25:1, or 0:1 to about 15:1, or 0:1 to about 5:1, or 1:1 to about 10:1, or about 1:1 to 5:1.

In some embodiments, the mole ratio of hydrogen to hydrocarbon is greater than 0:1 to about 0.1:1, the chloride concentration is about 100 ppm to about 5000 ppm, and the mole ratio of hydrogen to chloride is greater than 0:1 to about 100:1.

By selecting an appropriate combination of mole ratio of hydrogen to hydrocarbon feed, chloride concentration, and mole ratio of hydrogen to chloride for a given catalyst, the selectivity for disproportionation products can be at least about 5%, or at least about 10%, or at least about 15%, or at least about 20%, or at least about 25%, or at least about 30% or at least about 35%, or at least about 40%, or at least about 45%, or at least about 50%, or at least about 55%, or at least about 60%, or at least about 65%, or at least about 70%, or at least about 75%, or at least about 80%. The % selectivity for the disproportionation reaction is defined as: [(sum of the wt. % Cₓ₋ and Cₓ₊ compounds)/(100-wt. % Cₓ feed)]×100. When the feed contains n-Cₓ and i-Cₓ, part of the n-Cₓ or i-Cₓ can isomerize depending on the initial feed concentrations and the equilibrium constant. In the case where isomerization of n-Cₓ to i-Cₓ occurs, the % selectivity for the disproportionation reaction is (wt. % C₍ₓ₋₁₎₋ in product+wt. % C₍ₓ₊₁₎₋ in product−wt. % C₍ₓ₋₁₎₋ in feed−wt. % C₍ₓ₋₁₎₊ in feed)/(wt. % nCₓ in feed−wt. % nCₓ in product)×100. As the feed composition increases in complexity, a simple equation similar to these may not be adequate. The % selectivity for the Cₓ₊ disproportionation products is defined as: [(sum of the Cₓ₊ compounds)/(100−wt. % Cₓ feed)]×100 and can be at least about 5%, or at least about 10%, or at least about 15%, or at least about 20%, or at least about 25%.

For example, with an nC₅ feed, at a mole ratio of hydrogen to hydrocarbon of 0.02, a mole ratio of hydrogen to chloride of 5, and a chloride concentration of 1600 ppm, the selectivity for the disproportionation products was over 50%.

The hydrocarbon feed 105 in admixture with hydrogen 115 and the chloride promoter 120 contacts the disproportionation catalyst in a disproportionation reaction zone 110 to obtain disproportionation products. The hydrocarbon feed 105 can be pre-heated with heat exchangers 125 and 130 and heater 135, for example.

The disproportionation reaction zone 110 may be in a single reactor or two or more separate reactors with suitable heaters between them to ensure that the desired disproportionation temperature is maintained at the entrance to each reactor. The reactants may be contacted with the catalyst in upward, downward, or radial flow fashion. The reactants may be in the liquid phase, a mixed liquid-vapor phase, or a vapor phase when contacted with the catalyst, with excellent results being obtained with primarily liquid-phase operation. Contacting may be effected using the catalyst in a fixed-bed system, a moving-bed system, a fluidized-bed system, or in a batch-type operation.

As illustrated, the disproportionation reaction zone 110 includes two reactors 140, 145. The hydrocarbon feed 105 with the hydrogen 115 and chloride promoter 120 enters reactor 140 where it contacts the disproportionation catalyst. The effluent 150, which contains unreacted hydrocarbon feed and disproportionation reaction products, is sent to heat exchanger 130 to exchange heat with the incoming hydrocarbon feed 105 and then to reactor 145 where it contacts the disproportionation catalyst. Effluent 155, which contains unreacted hydrocarbon feed and disproportionation products from reactor 145 as well as from reactor 140, is sent to heat exchanger 125 and then to separation zone 160.

Suitable disproportionation reaction conditions include a temperature in the range of about 100° C. to about 300° C., or about 150° C. to about 300° C., or about 175° C. to about 300° C., or about 200° C. to about 300° C., or about 225° C. to about 300° C., or about 250° C. to about 300° C. The pressure is generally in the range of about 0 MPa (g) to about 13.8 MPa (g), or about 0 MPa (g) to about 10.0 MPa (g), or about 0 MPa (g) to about 7.5 MPa (g), or about 0 MPa (g) to about 5.0 MPa (g), or about 0 MPa (g) to about 3.5 MPa (g). The liquid hourly space velocity (LHSV) is generally in the range of about 0.25 hr⁻¹ to about 10 hr^{31 1}, or about 0.25 hr⁻¹ to about 7 hr⁻¹, or about 0.25 hr⁻¹ to about 5 hr⁻¹, or about 0.25 hr⁻¹ to about 3 hr⁻¹, or about 0.25 hr⁻¹ to about 2 hr⁻¹, or about 0.5 hr⁻¹ to about 2 hr⁻¹, or about 1 hr⁻¹ to about 2 hr⁻¹. The contacting time is in the range of a few seconds to hours, or about 0 5 min to about 10 hr, or about 0.5 min to about 8 hr, or about 0.5 min to about 6 hr, or about 0.5 min to about 4 hr, or about 0.5 min to about 2 hr, or about 0 5 min to about 1 hr, or about 1 min to about 1 hr, or about 5 min to about 1 hr.

The disproportionation reaction zone 110 contains a disproportionation catalyst. The catalyst is a solid acid catalyst comprising a refractory inorganic oxide having a metal halide dispersed thereon. There can optionally be a Group VIII metal component dispersed thereon.

Suitable refractory inorganic oxides include, but are not limited to, alumina, titania, zirconia, chromia, zinc oxide, magnesia, thoria, boria, silica, aluminum phosphate, silica-alumina, silica-magnesia, chromia-alumina, alumina-boria, silica-zirconia and other mixtures thereof.

Alumina is a suitable refractory inorganic oxide for use in the process. Suitable alumina materials are the crystalline aluminas known as gamma-, eta-, and theta-alumina. Zirconia, alone or in combination with alumina, comprises an alternative inorganic-oxide component of the catalyst. In some embodiments, the refractory inorganic oxide can have an apparent bulk density of about 0.3 to about 1.01 g/cc and surface area characteristics such that the average pore diameter is about 20 to 300 angstroms, the pore volume is about 0.05 to about 1 cc/g, and the surface area is about 50 to about 500 m²/g.

The alumina can be formed into any desired shape or type of carrier material known to those skilled in the art such as rods, pills, pellets, tablets, granules, extrudates, and like forms by methods well known to the practitioners of the catalyst material forming art. Spherical carrier particles may be formed, for example, from this alumina by: (1) converting the alumina powder into an alumina sol by reaction with a suitable peptizing acid and water and thereafter dropping a mixture of the resulting sol and a gelling agent into an oil bath to form spherical particles of an alumina gel which are easily converted to a gamma-alumina carrier material by known methods; (2) forming an extrudate from the powder by established methods and thereafter rolling the extrudate particles on a spinning disk until spherical particles are formed which can then be dried and calcined to form the desired particles of spherical carrier material; and (3) wetting the powder with a suitable peptizing agent and thereafter rolling the particles of the powder into spherical masses of the desired size.

The extrudate particle form of the carrier material may be prepared by mixing alumina powder with water and suitable peptizing agents such as nitric acid, acetic acid, aluminum nitrate, and the like material until an extrudable dough is formed. The amount of water added to form the dough is typically sufficient to give a Loss on Ignition (LOI) at 500° C. of about 30 to 65 mass %. The acid addition is generally sufficient to provide 2 to 7 mass % of the volatile-free alumina powder used in the mix. Preferably from about 0.1 to about 10 mass-% of an extrusion aid such as Methocel, and more preferably from about 1 to about 5 mass-%, is included in the mix. The resulting dough optimally is then mulled and extruded through a suitably sized die to form extrudate particles as described hereinabove.

The extrudate particles are dried at a temperature of about 150° C. to about 200° C., and then calcined at a temperature of about 400° C. to about 800° C. for a period of 0.2 to 10 hours to create the preferred form of the refractory inorganic oxide catalyst base. The calcination is typically effected within a temperature range of from of about 545° C. to about 610° C., or about 560° C. to about 580° C. In some embodiments, the calcination conditions are established to provide a finished-catalyst surface area of about 150 to about 280 m²/g (or or about 150 to about 230 m²/g) with an average pore diameter of from about 35 to about 60 angstroms.

Another component of the catalyst of the present invention is a metal halide, such as a Friedel-Crafts type metal halide. Suitable metal halides of the Friedel-Crafts type include aluminum chloride, aluminum bromide, ferric chloride, ferric bromide, zinc chloride and the like compounds, with the aluminum halides and particularly aluminum chloride ordinarily yielding the best results. Generally, this component can be incorporated into the catalyst using any of the conventional methods for adding metallic halides of this type. Good results are obtained when the metallic halide is sublimed onto the surface of the support. Further details concerning one method of sublimation are disclosed in U.S. Pat. No. 2,999,074, for example.

In some embodiments, when the calcined refractory inorganic-oxide support is loaded with a metal halide component, the presence of chemically combined hydroxyl groups in the refractory inorganic oxide allows a reaction to occur between the metal halide and the hydroxyl group of the support. For example, aluminum chloride reacts with the hydroxyl groups in the preferred alumina support to yield Al—O—AlCl₂ active centers which enhance the catalytic behavior of the catalyst. Since chloride ions and hydroxyl ions occupy similar sites on the support, more hydroxyl sites will be available for possible interaction with the metal halide when the chloride population of the sites is low. In some embodiments, the metal halide may be impregnated onto the catalyst by sublimation of the metal halide onto the calcined support under conditions to combine the sublimed metal halide with the hydroxyl groups of the calcined support. This reaction is typically accompanied by the elimination of about 0.5 to about 2.0 moles of hydrogen chloride per mole of metal halide reacted with the inorganic-oxide support. In subliming aluminum chloride, which sublimes at about 184° C., suitable loaded temperatures range from about 190° C. to 750° C., with a preferable range being from about 200° C. to 650° C. The sublimation can be conducted at atmospheric pressure or under increased pressure and in the presence or absence of diluent gases such a hydrogen or light paraffinic hydrocarbons or both. The impregnation of the metal halide may be conducted batch-wise. One preferred method for impregnating the calcined support is to pass sublimed AlCl₃ vapors, in admixture with a carrier gas such as hydrogen, through a calcined catalyst bed. This method both continuously deposits and reacts the aluminum chloride and also removes the evolved HCl.

The amount of metal halide combined with the calcined support may range from about 0.1 up to about 30 mass % to the metal-halide-free, calcined composite, or about 0.1 up to about 25 mass %, or about 0.1 up to about 20 mass %, or about 0.1 up to about 15 mass %. The final composite containing the sublimed Friedel-Crafts metal halide is treated to remove the unreacted metal halide by subjecting the composite to a temperature above the sublimation temperature of the metal halide for a time sufficient to remove from the composite any unreacted metal halide. In the case of AlCl₃, temperatures of about 400° C. to 650° C., and times of from about 1 to 48 hours are sufficient.

In some embodiments, the refractory inorganic oxide support can be pretreated with HCl to convert the Al—OH bonds to Al—Cl before loading with the AlCl₃.

An optional ingredient of the catalyst is a Group VIII metal component. Of the Group VIII metals, platinum group metals, e.g., platinum, palladium, rhodium, ruthenium, osmium and iridium, are preferred, particularly platinum. Mixtures of Group VIII metals can also be used. This component may exist within the final catalytic composite as a compound such as an oxide, sulfide, halide, or oxyhalide, in chemical combination with one or more of the other ingredients of the composite, or as an elemental metal. Best results are obtained when substantially all of this component is present in the elemental state. This component may be present in the final catalyst composite in any amount which is catalytically effective, but relatively small amounts are preferred. In fact, the surface-layer Group VIII metal component generally will comprise about 0.01 to 2 mass % of the final catalyst, calculated on an elemental basis. Excellent results are obtained when the catalyst contains about 0.05 to 1 mass % of platinum.

Typical platinum-group compounds which may be employed in preparing the catalyst of the invention are chloroplatinic acid, platinum dichloride, ammonium chloroplatinate, bromoplatinic acid, platinum tetrachloride hydrate, dicarbonylplatinum dichloride, dinitrodiaminoplatinum, palladium chloride, palladium chloride dihydrate, palladium nitrate, etc. Chloroplatinic acid is preferred as a source of the preferred platinum component. A surface-layer platinum component may be impregnated onto the catalyst from a solution of chloroplatinic acid in the absence of strong mineral acids such as hydrochloric and nitric acid.

In some embodiments, the platinum-group metal component is concentrated in the surface layer of each catalyst particle. A “surface-layer” component has a concentration in the micron surface layer of the catalyst particle that is at least 1.5 times the concentration in the central core of the catalyst particle. Preferably, the surface-layer concentration of platinum-group metal is at least about twice the concentration in the central core. As exemplified herein below, the surface layer may be 100 or 150 microns deep and the central core may be 50% of the volume or 50% of the diameter of the particle; however, other quantitative criteria are not excluded thereby. Further details of the characteristics and preparation of a surface-layer platinum-group metal component are contained in U.S. Pat. No. 5,004,859, for example.

The catalyst may contain other metal components known to modify the effect of the Group VIII metal component. Such metal modifiers may include rhenium, tin, germanium, lead, cobalt, nickel, indium, gallium, zinc, uranium, dysprosium, thallium, and mixtures thereof. Catalytically effective amounts of such metal modifiers may be incorporated into the catalyst by any means known in the art. In some embodiments, the catalyst consists essentially of the alumina support, a metal halide, and a platinum-group metal component. This formulation is free of modifier metals, such as tin or indium or halogen other than in the metal halide.

If a Group VIII metal is included, it can be added before the metal halide. In this case, the composite of the alumina and Group VIII metal is dried and calcined before addition to the metal halide. The drying is carried out at a temperature of about 100° C. to 300° C., followed by calcination or oxidation at a temperature of from about 375° C. to 600° C. in an air or oxygen atmosphere for a period of about 0.5 to 10 hours in order to convert the metallic components substantially to the oxide form.

In some embodiments, the resultant oxidized catalytic composite is subjected to a substantially water-free and hydrocarbon-free reduction step prior to its use in the conversion of hydrocarbons. This step is designed to selectively reduce the platinum-group metal component to the corresponding elemental metal and to insure a finely divided dispersion of the metal component throughout the carrier material. Preferably, substantially pure and dry hydrogen (i.e., less than 20 vol. ppm H₂O) is used as the reducing agent in this step. The reducing agent is contacted with the oxidized composite at conditions including a temperature of about 425° C. to about 650° C. and a period of time of about 0.5 to about 2 hours to reduce substantially all of the platinum-group component to its elemental metallic state. This reduction treatment may be performed in situ as part of a start-up sequence if precautions are taken to pre-dry the plant to a substantially water-free state and if substantially water-free and hydrocarbon-free hydrogen is used. Contact with water in general is to be avoided as water will deactivate the catalyst. Thus, both catalyst treatment and operation should be substantially water free.

The catalyst may contain an additional halogen component. The halogen component may be either fluorine, chlorine, bromine or iodine or mixtures thereof or an organic polyhalo component. Chlorine is the preferred halogen component. The halogen component is generally present in a combined state with the inorganic-oxide support. Although not essential to the invention, the halogen component is preferably well dispersed throughout the catalyst. The halogen component may comprise from more than 0.2 to about 15 mass-%, calculated on an elemental basis, of the final catalyst. Further details of halogen components and their incorporation into the catalyst are disclosed in U.S. Pat. No. 5,004,859 referenced above.

The catalyst can be characterized by a pore-acidity index, calculated as 100×(PD×Acidity/SA) wherein PD=average pore diameter in angstroms; Acidity=mmols TMP/g @ 120° C. and SA=surface area in m²/g. In some embodiments, the catalysts may have a pore-acidity index of at least about 7.0.

Surface area is measured using nitrogen by the well-known BET (Brunauer-Emmett-Teller) method, which also indicates average pore diameter. Acidity is measured by loading the sample as powder in a glass tube and pretreating under high vacuum (ca. 10⁻⁶ torr) at 600° C. for 2 hours. The samples are then cooled to 120° C. and exposed to trimethylphosphine (TMP) for 15 minutes followed by a 45-minute equilibration time, and then degassed with high vacuum. The TMP exposed to the sample is stored in a known volume of gas line and is exposed to the sample by opening a valve connecting this line to sample chamber. The amount of adsorbed TMP is calculated from the vapor-pressure drop caused by adsorption on the sample from the known volume of the gas line, compared to the change in vapor pressure with no sample present.

The effluent 155 from reactor 145 containing the disproportionation products and unreacted hydrocarbon feed is sent to a separation zone 160 where it is separated into at least two streams 165, 170. Suitable separation processes include, but are not limited to, distillation columns and adsorption processes.

In some embodiments, there can be one or more recycle streams 175 which can be combined with the hydrocarbon feed 105 and recycled to the disproportionation reaction zone 110.

For example, the disproportionation of C₅ paraffins can produce a stream of lighter C₄₋ paraffins which can be used as a feed for an alkylation unit and a stream of higher boiling C₆ paraffins, suitable for gasoline blinding or reformer feed. In some embodiments, there could also be a C₅ stream. At least a portion of the C₅ stream could be recycled to the disproportionation reaction zone 110. In one example, a light naphtha feed can be separated into a C₅ stream and a C₆ stream in a splitter (not shown). The C₆ stream could be sent to a reformer, a standard isomerization unit, or a sent directly to a gasoline stream. The C₅ stream from the splitter can be used as the hydrocarbon feed for the disproportionation reaction zone.

As another example, the disproportionation of iso-pentane (iso-C₅) produces products which can be separated into a C₄₋ stream and a C₅₊ stream. The C₄₋ stream contains isobutane (iso-C₄), which can be used as a feed for an alkylation process. The C₅₊ stream contains C₆₊ isoparaffins, which could be blended with gasoline. The RVP of the C₅₊ fraction product would be lower than the RVP of the iso-C₅ feed.

Since paraffin disproportionation is an equilibrium limited reaction, equilibrium amounts of C₅ will be present with the products; it is desirable to recycle the C₅ material to increase the C₅ conversion. This can be done by separating the effluent into at least a C₄₋ stream, a C₅ stream and a C₆₊ stream. The C₅ stream could be recycled to the disproportion reaction zone, and the C₆₊ stream could be used as gasoline blendstock.

In some embodiments, the separation could yield at least an iso-C₄₋ stream, an iso-C₅ stream, and an n-C₅₊ stream. In some embodiments, there could also be a separate n-C₄₋ stream, or the n-C₄₋ stream could be combined with the iso-C₄₋ stream. The stream of unconverted iso-C₅ could be recycled to the disproportionation reaction zone.

In some embodiments, the separation could result in at least an iso-C₄ stream, a C₅ stream comprising iso-C₅ and n-C₅, and an n-C₆₊ stream. In some embodiments, there could also be a separate n-C₄₋ stream, or the n-C₄₋ stream could be combined with the iso-C₄₋ stream. The C₅ stream could be recycled to the disproportionation reaction zone.

In some embodiments, the separation could yield at least an iso-C₄ stream, a stream comprising n-C₄ and iso-C₅, and an n-C₅₋ stream. The stream comprising n-C₄ and iso-C₅ could be recycled to the disproportionation reaction zone. Recycling the n-C₄ to the disproportionation reaction zone will result in it being isomerized to iso-C₄.

In some embodiments, light naphtha stream could be fed to the disproportionation reaction zone without being separated first.

When the hydrocarbon feed is a C₄ feed, the separation could yield at least a C₃₋ stream, a C₄ stream, and a C₅₋ stream. The C₄ stream could be recycled to the disproportionation reaction zone. In some embodiments, the C₄ stream could also be separated into an n-C₄₋ stream and an iso-C₄₋ stream.

With a C₇ feed, the separation could result in at least a C₆₋ stream, a C₇ stream, and a C₈₊-rich stream, with the C₇ stream being recycled to the disproportionation reaction zone.

Additional separations could be made as would be understood by those of skill in the art.

After a period of use, the disproportionation catalyst will become deactivated due to coke formation. The deactivated catalyst can be regenerated. Once the catalyst reaches a predetermined level of deactivation, the regeneration process could be initiated. The feed would be flushed from the disproportionation reaction zone. One method of regeneration involves heating the catalyst, desirably in the presence of hydrogen and optionally a hydrocarbon. The hydrocarbon has a higher heat capacity than hydrogen and can assist in increasing the reaction temperature within the reactor. Any suitable hydrocarbon can be used, including but not limited to, isobutane. The molar ratio of hydrogen to hydrocarbon is typically in the range of 1:20 to 20:1. In some embodiments, the catalyst can be heated to a temperature in the range of about 100° C. to about 300° C., or about 125° C. to about 275° C., or about 150° C. to about 250° C., or about 150° C. to about 225° C., or about 150° C. to about 200° C., or about 175° C. to about 300° C. The catalyst is typically heated for at least about 1 h, or in the range of about 0.25 to about 24 hr.

### EXAMPLES

### Example 1

### Catalyst

The catalyst is a chlorided alumina catalyst containing platinum made for example by U.S. Pat. No. 5,004,859. The concentration of platinum ranged from 0.002 wt. % to 2 wt. %, the chloride concentration ranged from 0.1 to 10 wt. % and the alumina phase was one of alpha, gamma, eta or theta.

### Example 2

### Experimental Set Up

The catalytic reactions were typically run using a ⅞″ inner diameter stainless steel tube reactor. Prior to catalyst loading, the reactor was dried by heating the reactor to at least 150° C. with a three-zone clam shell furnace under a stream of flowing nitrogen for at least four hours. After the drying procedure was completed, the reactor was cooled to ambient temperature, connected to a nitrogen line, and the reactor opened under flowing nitrogen. The reactor was inserted through a hole in a nitrogen glovebag, and the connection of the glovebag with the reactor was sealed with electrical tape. The top of the open reactor was enclosed within a glovebag and had nitrogen blowing through it. The catalyst from Example 1 was loaded under nitrogen in the glovebag to the reactor under this positive flow of nitrogen. The reactor was sand packed with 50-70 mesh sand, the sand having been previously calcined to 700° C. for 7 h. Typically, 40 ccs of catalyst was loaded into the reactor, and the reaction was run downflow. The feed had a 1.4 MPa(g) (210 psig) hydrogen header and the concentration of dissolved hydrogen in the feed was determined from the literature values reported in the IUPAC Solubility Data Series volumes ⅚ “Hydrogen and Deuterium” (1981) for pentane and butane. It was assumed that the value for pentane would remain constant for the iC5, iC5/nC5 and iC5/nC5/cyclopentane (CP) feeds. The feed was passed through a high surface sodium dryer prior to introduction to the reactor and was added to the reactor by means of a Quizzix pump. A second pump controlled the chloride addition rate. The chloride was dissolved in the feed, and the chloride source (2-chlorobutane) had previously been dried with activated 3A molecular sieves. The two feed streams were introduced to the reactor by joining the two separate feed streams with a Tee connector immediately prior to their introduction to the reactor. The temperature was measured using K-type thermocouples, and the pressure was controlled by means of a backpressure regulator. The effluent was sent directly to an Agilent 6890N gas chromatograph (GC), and the product was analyzed by means of flame ionization detection. A 60 m, 0.32 mm inner diameter, 1.0 μm film thickness DB-1 column was used. The initial oven temperature was 40° C., with a 4 minute hold time at this temperature. The oven was then ramped to 135° C. at a 5° C./min ramp rate, and the program was completed once this temperature reached. The GC inlet was 250° C. with a hydrogen carrier gas. The product was then sent directly to a product charger and collected.

### Example 3

### Disproportionation of iC5 with Regeneration

The catalytic reaction was run according to the procedure outlined above, except 30 ccs of catalyst was used and a header of nitrogen was present on the feed chargers. The conditions and results are listed in Table 1 below. After the catalyst had deactivated, the catalyst was regenerated by flushing the feed out of the reactor, purging the reactor with hydrogen and pressurizing with hydrogen to about 193 kPa (g) (28 psig) and then heating to 175° C. for about 2 h. The regeneration occurred after 30 h on stream. After the regeneration, the reactor was cooled to the desired temperature, the pressure was adjusted, and then feed was reintroduced to the system. The results are shown below in Table 1 and demonstrate that the disproportionation of iC5 occurs with this type of catalyst, but deactivates with time on stream (TOS).

TABLE 1
Disproportionation of iC5 and regenerations results

| TOS (h) | 12 | 21 | 30 | 47^{a} | 55^{a} |
|---|---|---|---|---|---|
| T (° C.) | 145 | 145 | 145 | 146 | 146 |
| P (psig) | 449 | 454 | 456 | 455 | 454 |
| Cl (ppm) | 1700 | 1600 | 1100 | 1600 | 1600 |
| LHSV (hr⁻¹) | 1.9 | 2.0 | 2.8 | 2.0 | 2.0 |
| H₂/HC ^{b} | 0.0 | 0.0 | 0.0 | 0.0 | 0.0 |
| H₂/Cl^{c} | 0.0 | 0.0 | 0.0 | 0.0 | 0.0 |
| % iC5 Conv.^{d} | 25 | 10 | 1 | 18 | 12 |
| % C5P Conv.^{e} | 18 | 7 | <1 | 13 | 10 |
| % Selec. Disp.^{f} | 72 | 74 | 42^{g} | 74 | 76 |
| Compound | | | | | |
| Methane | 0.00 | 0.00 | 0.00 | 0.00 | 0.00 |
| Ethane | 0.00 | 0.00 | 0.00 | 0.00 | 0.00 |
| Propane | 0.04 | 0.01 | 0.00 | 0.02 | 0.01 |
| iC4 | 7.38 | 2.99 | 0.17 | 5.26 | 3.90 |
| nC4 | 0.37 | 0.13 | 0.01 | 0.16 | 0.14 |
| iC5 | 74.83 | 89.89 | 98.82 | 82.45 | 87.44 |
| nC5 | 6.93 | 2.65 | 0.69 | 4.63 | 3.00 |
| Neopentane | 0.02 | 0.01 | 0.01 | 0.01 | 0.01 |
| 22DMB | 0.27 | 0.04 | 0.00 | 0.12 | 0.06 |
| 23DMB | 1.33 | 0.54 | 0.03 | 0.98 | 0.70 |
| 2MP | 4.42 | 2.02 | 0.14 | 3.43 | 2.63 |
| 3MP | 2.57 | 1.17 | 0.08 | 1.99 | 1.52 |
| nC6 | 0.71 | 0.15 | 0.00 | 0.36 | 0.20 |
| C7+ | 1.14 | 0.39 | 0.04 | 0.56 | 0.38 |
| Unknown | 0.00 | 0.01 | 0.00 | 0.02 | 0.00 |

^{a}After the regeneration procedure,
^{b} molar ratio of hydrogen to hydrocarbon in feed,
^{c}molar ratio of hydrogen to chloride,
^{d}% iC5 Conv. = 100 − wt. % iC5,
^{e}% C5P Conv. = 100 − wt. % iC5 − wt. % nC5,
^{f}% Selec. Disp. = (wt. % C₄₋ + wt. % C₆₊)/(100 − wt. % iC5) × 100 and
^{g}selectivity may be off due to increased error from low conversion and the assumption of exactly 100 wt. % iC5.

### Example 4

### Disproportionation of iC5

The catalytic reaction was run according to the procedure outlined above. The conditions and results are listed in Table 2 below and demonstrate that the presence of small amounts of hydrogen increase the stability of the catalyst.

TABLE 2
Disproportionation of iC5

| TOS (h) | 15 | 20 | 25 |
|---|---|---|---|
| T (° C.) | 172 | 172 | 172 |
| P (psig) | 608 | 610 | 611 |
| Cl (ppm) | 1600 | 1600 | 1600 |
| LHSV (hr⁻¹) | 1.0 | 1.0 | 1.0 |
| H₂/HC ^{a} | 0.02 | 0.02 | 0.02 |
| H₂/Cl^{b} | 5 | 5 | 5 |
| % iC5 Conv.^{c} | 52 | 53 | 54 |
| % C5P Conv.^{d} | 41 | 42 | 42 |
| % Selec. Disp. ^{e} | 80 | 80 | 77 |
| Compound | | | |
| Methane | 0.00 | 0.00 | 0.00 |
| Ethane | 0.00 | 0.00 | 0.00 |
| Propane | 0.53 | 0.62 | 0.63 |
| iC4 | 21.07 | 21.67 | 20.18 |
| nC4 | 2.54 | 2.54 | 2.51 |
| iC5 | 48.28 | 47.06 | 45.62 |
| nC5 | 10.51 | 10.89 | 12.36 |
| 22DMB | 1.06 | 1.08 | 1.34 |
| 23DMB | 1.77 | 1.75 | 1.79 |
| 2MP | 5.81 | 5.73 | 5.91 |
| 3MP | 3.45 | 3.41 | 3.55 |
| nC6 | 1.49 | 1.47 | 1.63 |
| C7P | 2.32 | 2.42 | 2.45 |
| C8+ | 1.17 | 1.37 | 1.40 |
| Unknown | 0.00 | 0.00 | 0.00 |

^{a} Molar ratio of hydrogen to hydrocarbon in feed,
^{b}molar ratio of hydrogen to chloride,
^{c}% iC5 Conv. = 100 − wt. % iC5,
^{d}% C5P Conv. = 100 − wt. % iC5 − wt. % nC5 and
^{e} % Selec. Disp. = (wt. % C₄₋ + wt. % C₆₊)/(100 − wt. % iC5) × 100.

### Example 5

### Disproportionation of nC5

The catalytic reaction was run according to the procedure outlined above. The conditions and results are listed in Table 3 below and demonstrate that the disproportionation of nC5 readily occurs with these types of catalysts and that with small amounts of hydrogen being present, the catalyst stability is increased.

TABLE 3
Disproportionation of nC5

| TOS (h) | 8 | 13 | 28 |
|---|---|---|---|
| T (° C.) | 176 | 175 | 171 |
| P (psig) | 619 | 618 | 622 |
| Cl (ppm) | 1600 | 1600 | 1600 |
| LHSV (hr⁻¹) | 1.0 | 1.0 | 1.0 |
| H₂/HC ^{a} | 0.02 | 0.02 | 0.02 |
| H₂/Cl^{b} | 5 | 5 | 5 |
| % nC5 Conv.^{c} | 69 | 68 | 67 |
| % C5P Conv.^{d} | 35 | 35 | 33 |
| % Selec. Disp. ^{e} | 50 | 52 | 50 |
| Compound | | | |
| Methane | 0.00 | 0.00 | 0.00 |
| Ethane | 0.00 | 0.00 | 0.00 |
| Propane | 0.81 | 0.81 | 0.66 |
| iC4 | 15.49 | 15.83 | 15.19 |
| nC4 | 3.70 | 3.32 | 2.54 |
| iC5 | 34.05 | 32.70 | 33.60 |
| nC5 | 31.26 | 32.06 | 33.18 |
| 22DMB | 1.47 | 1.35 | 1.33 |
| 23DMB | 1.30 | 1.36 | 1.37 |
| 2MP | 4.09 | 4.25 | 4.25 |
| 3MP | 2.54 | 2.64 | 2.62 |
| nC6 | 1.44 | 1.45 | 1.32 |
| C7P | 2.46 | 2.64 | 2.44 |
| C8+ | 1.39 | 1.60 | 1.50 |
| Unknown | 0.00 | 0.00 | 0.00 |

^{a} Molar ratio of hydrogen to hydrocarbon in feed,
^{b}molar ratio of hydrogen to chloride,
^{c}% nC5 Conv. = 100 − wt. % nC5,
^{d}% C5P Conv. = 100 − wt. % iC5 − wt. % nC5 and
^{e} % Selec. Disp. = (wt. % C₄₋ + wt. % C₆₊)/(100 − wt. % nC5) × 100.

### Example 6

### Disproportionation of Pentanes in the Presence of Cyclopentane (CP)

The catalytic reaction was run according to the procedure outlined above. For the first part of the reaction, the feed was a blend of iC5/nC5. Once the reactivity of the paraffinic feed in the absence of significant amounts of naphthenes was established, a new feed was introduced comprising iC5/nC5/CP. The conditions and results are listed in Table 4 below and demonstrate that disproportionation readily occurs in the feed without CP, but upon CP introduction the activity for paraffin disproportionation decreases and the paraffin isomerization activity increases.

TABLE 4
Disproportionation of pentanes in the presence and absence of cyclopentane

| Feed | iC5/nC5 | iC5/nC5 | iC5/nC5 | iC5/nC5/CP | iC5/nC5/CP | iC5/nC5/CP |
|---|---|---|---|---|---|---|
| TOS (h) | 0 | 10 | 25 | 0 | 41 | 51 |
| T (° C.) | NA | 170 | 168 | NA | 166 | 165 |
| P (psig) | NA | 454 | 454 | NA | 452 | 451 |
| Cl (ppm) | NA | 1600 | 1600 | NA | 1600 | 1600 |
| LHSV (hr⁻¹) | NA | 1.0 | 1.0 | NA | 1.0 | 1.0 |
| H₂/HC^{a} | NA | 0.02 | 0.02 | NA | 0.02 | 0.02 |
| H₂/Cl^{b} | NA | 5 | 5 | NA | 5 | 5 |
| % nC5 | NA | 55 | 52 | NA | 56 | 56 |
| Conv.^{c} | | | | | | |
| % C5P | NA | 27 | 28 | NA | 14 | 15 |
| Conv.^{d} | | | | | | |
| % Selec. | NA | 96^{e} | 100^{f} | NA | 51^{e} | 53^{e} |
| Disp. | | | | | | |
| Compound | Feed | | | Feed | | |
| Methane | 0.00 | 0.01 | 0.00 | 0.00 | 0.00 | 0.00 |
| Ethane | 0.00 | 0.01 | 0.01 | 0.00 | 0.01 | 0.01 |
| Propane | 0.00 | 0.39 | 0.77 | 0.00 | 0.11 | 0.11 |
| iC4 | 0.02 | 11.58 | 12.96 | 0.01 | 6.52 | 6.62 |
| nC4 | 0.02 | 2.05 | 3.13 | 0.02 | 0.93 | 0.93 |
| iC5 | 49.18 | 50.28 | 47.19 | 40.35 | 51.89 | 51.29 |
| nC5 | 50.13 | 22.31 | 24.05 | 41.72 | 18.34 | 18.25 |
| 22DMB | 0.00 | 1.40 | 1.43 | 0.31 | 0.80 | 0.83 |
| 23DMB | 0.07 | 1.27 | 1.04 | 0.24 | 0.63 | 0.66 |
| 2MP | 0.11 | 4.04 | 3.29 | 0.47 | 2.04 | 2.12 |
| 3MP | 0.08 | 2.48 | 2.02 | 0.13 | 1.12 | 1.16 |
| nC6 | 0.26 | 1.22 | 1.10 | 0.29 | 0.62 | 0.65 |
| C7P | 0.08 | 1.72 | 1.76 | 0.03 | 0.99 | 1.00 |
| C8+ | 0.03 | 1.26 | 1.23 | 0.04 | 2.19 | 2.39 |
| Unknown | 0.00 | 0.00 | 0.00 | 0.00 | 0.00 | 0.00 |
| CP^{g} | 0.00 | 0.00 | 0.00 | 16.26 | 13.83 | 13.97 |

^{a}Molar ratio of hydrogen to hydrocarbon in feed,
^{b}molar ratio of hydrogen to chloride,
^{c}% nC5 Conv. = (wt. % nC5 in feed − wt. % nC5 in product)/(wt. % nC5 in feed) × 100,
^{d}% C5P Conv. = (wt. % nC5 in feed + wt. % iC5 in feed − wt. % nC5 in product − wt. % iC5 in product)/(wt. % nC5 in feed + wt. % iC5 in feed) × 100,
^{e}% Selec. Disp. = (wt. % C₄₋ in product + wt. % C₆₊ in product − wt. % C₄₋ in feed − wt. % C₆₊ in feed)/(wt. % nC5 in feed − wt. % nC5 in product) × 100,
^{f}% Selec. Disp. = 100 since net loss of both iC5 and nC5 and
^{g}CP overlapped with 23DMB in the GC, the concentration was estimated by quantifying the peak area for 22DMB and back-calculating the concentration for 23DMB assuming a 22DMB/23DMB ratio of 1.27 and then subtracting that value from the overlapped signal to arrive at the estimated CP concentration.

### Example 7

### Disproportionation of nC4

The catalytic reaction was run according to the procedure outlined above. The conditions and results are listed in Table 5 below and demonstrate that the disproportionation of nC4 readily occurs with these types of catalysts and that with small amounts of hydrogen being present, the catalyst is stable.

TABLE 5
Disproportionation of nC4

| TOS (h) | 0 | 18 | 40 |
|---|---|---|---|
| T (° C.) | NA | 165 | 165 |
| P (psig) | NA | 1036 | 1036 |
| Cl (ppm) | NA | 1600 | 1600 |
| LHSV (hr⁻¹) | NA | 1 | 1 |
| H₂/HC ^{a} | NA | 0.02 | 0.02 |
| H₂/Cl^{b} | NA | 6 | 6 |
| % nC4 Conv.^{c} | NA | 66 | 66 |
| % C4P Conv.^{d} | NA | 20 | 21 |
| % Selec. Disp. ^{e} | NA | 30 | 31 |
| Compound | | | |
| Methane | 0.00 | 0.11 | 0.10 |
| Ethane | 0.00 | 0.16 | 0.16 |
| Propane | 0.01 | 9.00 | 9.23 |
| iC4 | 0.16 | 45.98 | 45.54 |
| nC4 | 99.37 | 33.45 | 33.34 |
| iC5 | 0.00 | 7.15 | 7.35 |
| nC5 | 0.01 | 2.24 | 2.32 |
| 22DMB | 0.00 | 0.45 | 0.48 |
| 23DMB | 0.00 | 0.15 | 0.16 |
| 2MP | 0.00 | 0.47 | 0.51 |
| 3MP | 0.00 | 0.27 | 0.29 |
| nC6 | 0.30 | 0.19 | 0.20 |
| C7P | 0.04 | 0.21 | 0.15 |
| C8+ | 0.07 | 0.16 | 0.15 |
| Unknown | 0.03 | 0.00 | 0.00 |

^{a} Molar ratio of hydrogen to hydrocarbon in feed,
^{b}molar ratio of hydrogen to chloride,
^{c}% nC4 Conv. = ((wt. % nC4 in feed − wt. % nC4 in product)/wt. % nC4 in feed) × 100,
^{d}% C4P Conv. = ((wt. % iC4 in feed + wt. % nC4 in feed − wt. % iC4 in product − wt. % nC4 in product)/(wt. % iC4 in feed + wt. % nC4 in feed)) × 100 and
^{e} % Selec. Disp. = ((wt. % C₃₋ in product + wt. % C₅₊ in product − wt. % C₃₋ in feed − wt. % C₅₊ in feed)/(wt. % nC4 in feed − wt. % nC4 in product) × 100

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed:

1. A hydrocarbon disproportionation process comprising:
contacting a hydrocarbon feed in a disproportionation reaction zone with a disproportionation catalyst in the presence of hydrogen and an added chloride promoter under disproportionation conditions to obtain disproportionation products, wherein the disproportionation catalyst comprises a solid catalyst comprising a refractory inorganic oxide having a metal halide dispersed thereon.

2. The process of claim 1 wherein the disproportionation catalyst further comprises a Group VIII metal component dispersed thereon.

3. The process of claim 1 wherein the hydrogen is present in a mole ratio of hydrogen to hydrocarbon feed of greater than 0:1 to about 0.5:1.

4. The process of claim 1 wherein the chloride concentration from the added chloride promoter is in a range of greater than 0 to about 5000 ppm and a mole ratio of hydrogen to chloride from the added chloride promoter is in a range of greater than 0:1 to about 5000:1.

5. The process of claim 1 wherein a selectivity for disproportionation is at least about 25%.

6. The process of claim 1 wherein the hydrocarbon feed comprises alkanes having 4 to 7 carbon atoms.

7. The process of claim 1 wherein the disproportionation conditions include at least one of: a temperature in a range of about 100° C. to about 250° C., a pressure in a range of about 0 MPa (g) to about 13.8 MPa (g), and a liquid hourly space velocity of about 0.25 hr⁻¹ to about 10 hr⁻¹.

8. The process of claim 1 wherein the added chloride promoter comprises carbon tetrachloride, tetrachloroethylene, propyldichloride, butylchloride, chloroform, 2-chloro-2-methylpropane, 2-chloropropane, 2-chloro-2-methylbutane, 2-chloropentane, 1-chlorohexane, 3-chloro-3-methylpentane, 2-chlorobutane, or combinations thereof.

9. The process of claim 1 further comprising:
separating an effluent from the disproportionation reaction zone into at least two streams, the effluent containing the disproportionation products.

10. The process of claim 9 wherein the hydrocarbon feed comprises a C₅ feed, and wherein separating the effluent from the disproportionation reaction zone into the at least two streams comprises separating the effluent from the disproportionation reaction zone into at least an iso-C₄ stream, an iso-C₅ stream, and a n-C₅₊ stream; and further comprising:
optionally recycling at least a portion the iso-C₅ stream to the disproportionation reaction zone.

11. The process of claim 9 wherein the hydrocarbon feed comprises a C₅ feed, and wherein separating the effluent from the disproportionation reaction zone into the at least two streams comprises separating the effluent from the disproportionation reaction zone into at least an iso-C₄ stream, a C₅ stream comprising iso-C₅ and n-C₅, and a C₆₊ stream; and further comprising:
optionally recycling at least a portion of the C₅ stream to the disproportionation reaction zone.

12. The process of claim 9 wherein the hydrocarbon feed comprises a C₅ feed, and wherein separating the effluent from the disproportionation reaction zone into the at least two streams comprises separating the effluent from the disproportionation reaction zone into at least an iso-C₄ stream, a n-C₄ and iso-C₅ stream, and a n-C₅₊ stream; and further comprising:
optionally recycling at least a portion of the n-C₄ and iso-C₅ stream to the disproportionation reaction zone.

13. The process of claim 9 wherein the hydrocarbon feed comprises a C₄ feed, and wherein separating the effluent from the disproportionation reaction zone into the at least two streams comprises separating the effluent from the disproportionation reaction zone into at least a C₃₋ stream, a C₄ stream, and a C₅₊ stream; and further comprising:
optionally recycling at least a portion of the C₄ stream to the disproportionation reaction zone.

14. The process of claim 9 wherein the hydrocarbon feed comprises a C₇ feed, and wherein separating the effluent from the disproportionation reaction zone into the at least two streams comprises separating the effluent from the disproportionation reaction zone into at least a C₆₋ stream, a C₇ stream, and a C₈₊-rich stream; and further comprising:
optionally recycling at least a portion of the C₇ stream to the disproportionation reaction zone.

15. The process of claim 1 further comprising:
separating a light naphtha feed comprising C₅ and C₆ hydrocarbons into a C₅ stream and a C₆ stream: and wherein contacting the hydrocarbon feed in the disproportionation reaction zone with the disproportionation catalyst comprises contacting the C₅ stream in the disproportionation reaction zone with the disproportionation catalyst.

16. The process of claim 1 further comprising regenerating the disproportionation catalyst.

17. The method of claim 16 wherein regenerating the disproportionation catalyst comprises heating the disproportionation catalyst to a temperature in a range of about 100° C. to about 300° C. in the presence of hydrogen.

18. A hydrocarbon disproportionation process comprising:
contacting a hydrocarbon feed comprising alkanes having 4 to 7 carbon atoms in a disproportionation reaction zone with a disproportionation catalyst and in the presence of hydrogen and an added chloride promoter under disproportionation conditions including at least one of: a temperature in a range of about 100° C. to about 300° C., a pressure in a range of about 0 MPa (g) to about 13.8 MPa (g), and a liquid hourly space velocity of about 0.25 hr⁻¹ to about 10 hr⁻¹ to obtain disproportionation products;
wherein the disproportionation catalyst comprises a solid catalyst comprising a refractory inorganic oxide having a metal halide dispersed thereon and optionally a Group VIII metal dispersed thereon;
wherein the hydrogen is present in a mole ratio of hydrogen to hydrocarbon feed of greater than 0:1 to about 0.1:1;
wherein the added chloride promoter is present in an amount of at least about 100 ppm;
wherein a mole ratio of hydrogen to chloride is in a range of greater than 0:1 to about 100:1;
separating an effluent from the disproportionation reaction zone into at least two streams, the effluent containing the disproportionation products;
recovering at least one stream; and
optionally recycling at least a portion of one stream to the disproportionation reaction zone.

19. The method of claim 18 further comprising:
regenerating the disproportionation catalyst by heating the disproportionation catalyst to a temperature in a range about 100° C. to about 300° C. in the presence of hydrogen.

20. The process of claim 18 wherein the feed comprises a C₅ feed, and wherein separating the effluent from the disproportionation reaction zone into the at least two streams comprises separating the effluent from the disproportionation reaction zone into at least an iso-C₄ stream, an n-C₄ and iso-C₅ stream, and a n-C₅₊ stream; and wherein optionally recycling the at least one portion of one stream to the disproportionation reaction zone comprises optionally recycling the n-C₄ and iso-C₅ stream to the disproportionation reaction zone.
